(19) United States
(12) Patent Application Publication
(10) Pub. No.: US 2016/0100754 A1
(43) Pub. Date: Apr. 14, 2016

(54) OPHTHALMIC APPARATUS AND OPHTHALMIC APPARATUS CONTROL METHOD

(71) Applicant: CANON KABUSHIKI KAISHA, Tokyo (JP)

(72) Inventor: Yasuhiro Dobashi, Matsudo-shi (JP)

(21) Appl. No.: 14/877,108

(22) Filed: Oct. 7, 2015

(30) Foreign Application Priority Data
Oct. 10, 2014 (JP) 2014-208722

(51) Int. Cl. A61B 3/00; A61B 3/15; A61B 3/12

(52) U.S. Cl. CPC A61B 3/0025; A61B 3/12; A61B 3/0033; A61B 3/0041; A61B 3/154; A61B 3/152

(57) ABSTRACT

An ophthalmic apparatus includes a display control unit configured to cause a display unit to display a fundus image of an eye to be inspected, which is obtained by photographing an image of a fundus of the eye to be inspected by using a photographing optical system, an input unit configured to input whether to store the displayed fundus image, and a selecting unit configured to select, if it is input that the display fundus image is not to be stored, whether to execute rephotograph after automatically controlling a driving unit for driving at least one of a focusing portion formed in the photographing optical system and a photographing unit including the photographing optical system, or to execute rephotograph after manually controlling the driving unit.

### BACKGROUND OF THE INVENTION

1. Field of the Invention

The present invention relates to an ophthalmic apparatus such as a fundus camera to be used to observe the fundus of an eye to be inspected and photograph an image of the fundus in an ophthalmic hospital or the like, and a control method of the apparatus.

2. Description of the Related Art

Conventionally, fundus image photographing performed by a non-mydriatic fundus camera using infrared light in alignment has widely been used for the purposes of screening by mass examination and diagnosis of an eye disease.

Generally, various eye-to-be-inspected models are assumed when designing a fundus camera, and the camera is so designed as to have a sufficient margin for these models. However, an actual eye to be inspected sometimes has variations more than expected compared to the model eyes due to the influence of a disease or the like. In this case, it may be impossible to obtain a fundus image satisfactory for diagnosis if a normal image photographing method is used. In addition, a miotic pupil of an eye to be inspected must be prevented when using the non-mydriatic fundus camera. Therefore, observation is performed using infrared light having a wavelength different from that of visible light for photographing a fundus image, or the camera and an eye to be inspected are aligned by using infrared light.

Image photograph using the fundus camera produces a time difference between the timing at which an operator presses a release switch to the timing at which an image photographing light source emits flashlight. If a person to be inspected blinks his or her eyes during this time difference, this blink intercepts a light beam from the fundus, and excess reflected light is sometimes obtained from the anterior ocula segment. Also, if an eye to be inspected moves from a proper alignment position at the moment of image photograph, flare occurs in the periphery of a fundus image. Japanese Patent Application Laid-Open No. H05-015494 has disclosed a method of determining the quality of an image in accordance with the detection result of excess reflected light corresponding to a blink of a person to be inspected or flare, and automatically performing rephotograph if it is determined that an imaging error has occurred due to the blink or flare. In this case, even when the cause of the imaging error is an inappropriate alignment before the operator presses the release switch, automatic image photograph is performed again under the same conditions. Accordingly, there is the possibility that an error image similar to that photographed in the initial imaging is obtained in the rephotograph as well.

Also, Japanese Patent Application Laid-Open No. H08-275921 has disclosed an ophthalmic image photographing apparatus having automatic (auto) functions of automatically performing alignment and focusing of an eye to be inspected instead of an operator. This apparatus performs auto alignment and auto focusing by observing the reflected light of an index emitted on an eye to be inspected as an index image.

If the automatic functions of the apparatus are used when the curvature of the fundus or cornea of an eye to be inspected is larger or smaller than an expected value (the expectation of the apparatus), error images containing the same flare or defocus may be photographed regardless of how many times the image photograph is performed. As described above, when performing rephotograph due to an imaging error, it is sometimes undesirable to perform automatic image photograph under the same conditions again.

### SUMMARY OF THE INVENTION

The present invention reduces the reoccurrence of an imaging error when performing rephotograph due to the imaging error.

To solve the above problem, an ophthalmic apparatus according to the present invention includes a display control unit configured to cause a display unit to display a fundus image of an eye to be inspected, which is obtained by photographing an image of a fundus of the eye to be inspected by using a photographing optical system,

an input unit configured to input whether to store the displayed fundus image, and

a selecting unit configured to select, if it is input that the displayed fundus image is not to be stored, whether to execute rephotograph after automatically controlling a driving unit for driving at least one of a focusing portion formed in the photographing optical system and a photographing unit including the photographing optical system, or to execute rephotograph after manually controlling the driving unit.

The present invention can reduce the reoccurrence of an imaging error when performing rephotograph due to the imaging error, because photograph can be performed under different conditions.

Further features of the present invention will become apparent from the following description of exemplary embodiments with reference to the attached drawings.

### BRIEF DESCRIPTION OF THE DRAWINGS

FIG. 1 is a view showing an outline of the arrangement of a fundus camera according to the first embodiment.

FIG. 2 is a view schematically showing the optical arrangement of the fundus camera according to the first embodiment.

FIGS. 3A and 3B are schematic views each for explaining alignment using a prism in the first embodiment.

FIGS. 4A and 4B are schematic views each for explaining the contents of focusing.

FIG. 5 is a flowchart for explaining the steps of fundus photograph according to the first embodiment.

FIG. 6 shows a screen display example after photograph for use in the first embodiment.

FIG. 7 is a flowchart for explaining the steps of fundus photograph according to the second embodiment.

FIG. 8 shows a screen display example after photograph for use in the second embodiment.

FIG. 9 is a schematic view for explaining a mechanism of causing flare to be used in alignment correction.

FIGS. 10A, 10B and 10C are schematic views each for explaining a mechanism of causing defocus to be used in focus correction.

### DESCRIPTION OF THE EMBODIMENTS

### First Embodiment

The present invention will be explained in detail based on the embodiments shown in the accompanying drawings.

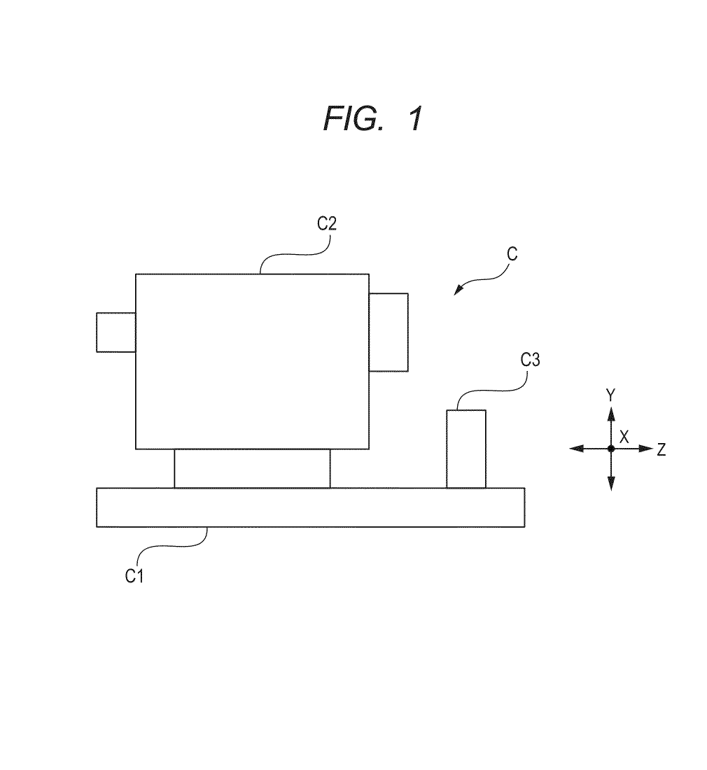

FIG. 1 is a view showing the arrangement of a fundus camera according to an embodiment as an ophthalmic apparatus. A fundus camera C includes a base C1, photographing unit C2, and joystick C3. The photographing unit C2 accommodates an optical system movable in the right-and-left direction (the X direction), the back-and-forth direction (the working distance, the Z direction), and the vertical direction (the Y direction) with respect to the base C1.

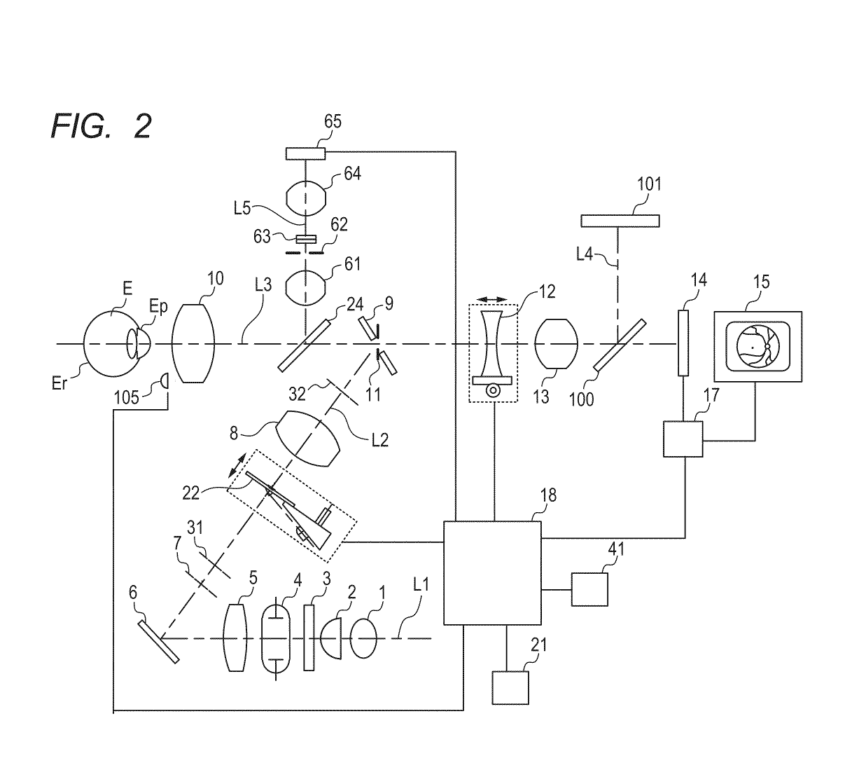

The photographing unit C2 is driven in the three-dimensional (XYZ) directions with respect to an eye E to be inspected (see FIG. 2) by a driving unit such as a motor formed in the base C1. The photographing unit C2 can also be moved in the XYZ directions with respect to the base C1 by operating the joystick C3.

Next, the optical system arranged in the photographing unit C2 of the fundus camera C will be explained with reference to FIG. 2. This optical system is a photographing optical system for observing an eye to be inspected and sensing or photographing an image of the eye. Note that the arrangement of this optical system will be explained in order for each of optical axes L1 to L5 on which various optical elements are arranged.

A light source which emits light for imaging or observing the fundus and elements for imaging or observing are arranged on the optical axis L1, and the optical axis L1 is formed along the optical path of the light emitted from the light source. On the optical axis L1, a condenser lens 2, a filter 3 for transmitting infrared light and blocking visible light, a photographing light source 4 such as a strobe, a lens 5, and a mirror 6 are arranged in this order from an observation light source 1 such as a halogen lamp which emits stationary light. On the optical axis L2 in the reflecting direction of the mirror 6, a ring stop 7 having a ring-like aperture, a crystal baffle 31, a relay lens 8, a corneal baffle 32, and a perforated mirror 9 having a central aperture are arranged in this order from the mirror 6.

In addition, on the optical axis L3 in the reflecting direction of the perforated mirror 9, a dichroic mirror 24 and an objective lens 10 opposing the eye E to be inspected are arranged in this order. The dichroic mirror 24 is insertable into and removable from the optical axis L3. A photographing stop 11 is arranged in the aperture of the perforated mirror 9, and a focusing lens 12 which adjusts the focus by moving on the optical axis L3, a photographing lens 13, and a half mirror 100 are sequentially arranged behind the photographing stop 11. An image sensor 14 which performs both moving image observation and still photograph is arranged beyond the half mirror 100. An internal fixation light 101 is arranged beyond the optical axis L4 as the reflecting direction of the half mirror 100.

The output image from the image sensor 14 is synthesized with a prepared character image by an image processor 17, and displayed on a monitor 15. The image processor 17 is also connected to a system controller 18, so the system controller 18 can analyze a fundus image transmitted from the image processor 17.

The system controller 18 controls the whole system, and can analyze the output image from each image sensor and perform automatic position control and focus control (to be described later). In addition, an operational input unit 21 operable by an examiner is connected to the system controller 18, and the next operation is determined by signal input from the examiner. The operational input unit 21 includes an input unit using the joystick C3 (to be described later), a photograph start button, a selection button, and the like. Furthermore, a storage memory 41 for storing photographed still images is connected to the system controller.

The arrangements of optical systems for use in alignment and focusing will be explained below.

First, the arrangement of an anterior ocula segment observation optical system for alignment will be explained.

On the optical axis L5 in the reflecting direction of the dichroic mirror 24, a lens 61, a stop 62, a prism 63, a lens 64, and a two-dimensional image sensor 65 having infrared sensitivity are arranged in this order. These elements form the anterior ocula segment observation optical system for observing an anterior ocular segment. In this system, light having entered the prism 63 is separated as it is refracted in opposite directions in the right-and-left direction in the upper and lower halves of the prism 63. Therefore, when the distance between the eye E to be inspected and the photographing unit C2 is longer than the appropriate working distance, the lens 61 forms an observation image of the anterior ocula segment in a position closer to the lens 61 than the prism 63, such that the upper half of the observation image is photographed on the right side, and the lower half thereof is photographed on the left side. When the distance between the eye E to be inspected and the photographing unit C2 is shorter than the appropriate working distance, the observation image is photographed such that the upper and lower halves are shifted to the opposite sides.

The anterior ocula segment of the eye E to be inspected is illuminated by an anterior ocula segment observation light source 105 having an infrared wavelength different from wavelengths transmitted through the filter 3 which blocks visible light. The above-described anterior ocula segment observation optical system can detect the alignment state between the eye E to be inspected and the anterior ocula segment. Note that the amount of misalignment is obtained by the above arrangement. This arrangement and a driving unit which is arranged in the base C1 described above and drives the photographing unit C2 in the three-axis directions form an alignment unit for controlling alignment of the photographing optical system with respect to an eye to be inspected in this embodiment.

The arrangement of the focusing optical system will now be explained.

A focusing index projector 22 is arranged between the ring stop 7 and relay lens 8 on the optical axis L2. This focusing index projector 22 projects split indices on a pupil Ep of the eye E to be inspected. The focusing index projector 22 and focusing lens 12 move in the directions of the optical axes L2 and L3, respectively, in synchronism with each other under the control of the system controller 18. In this state, the focusing index projector and image sensor 14 have an optically conjugate relationship. This focusing optical system can detect the focusing state of a fundus Er of the eye E to be inspected.

Note that the aforementioned arrangement obtains a defocus shift of the photographing optical system from an eye to be inspected. In this embodiment, this arrangement forms a focusing unit for obtaining the focused state of the photographing optical system with respect to an eye to be inspected. Also, in this embodiment, the above-descried driving unit drives a focusing portion (the focusing lens) in the focusing unit.

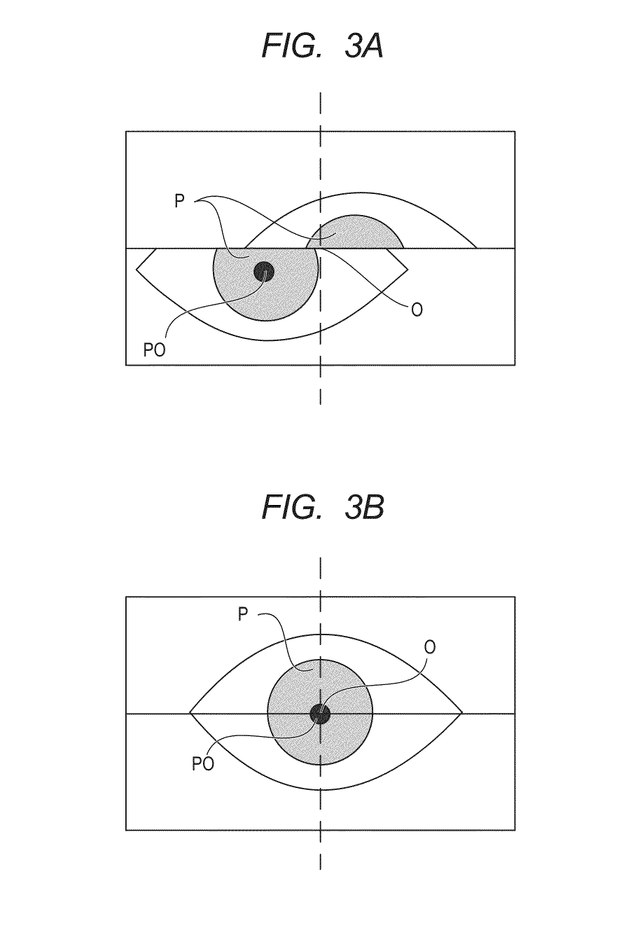

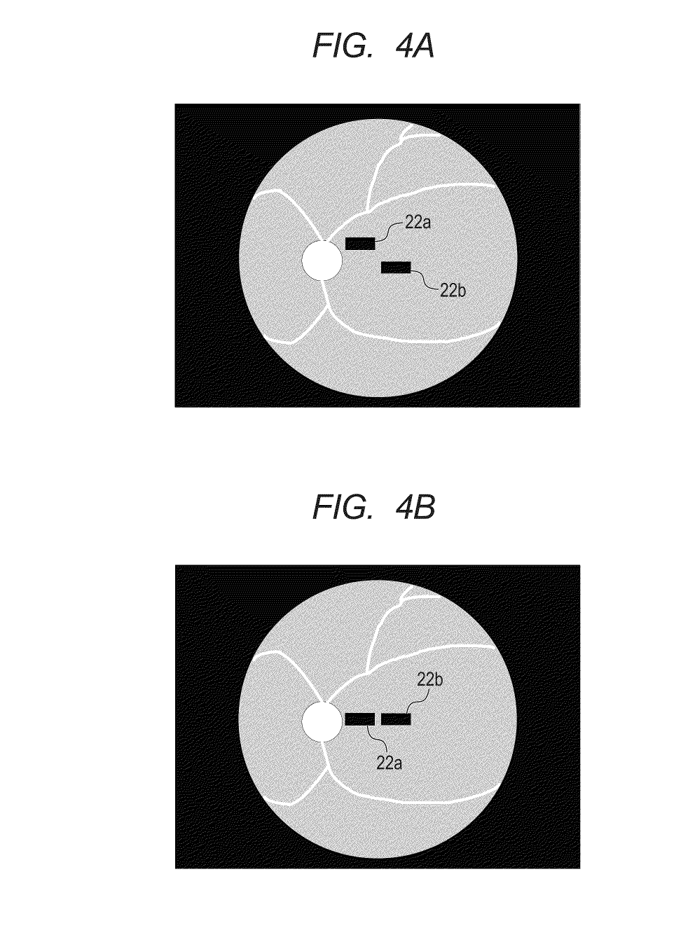

The arrangements of the optical systems for performing alignment and focusing have been explained above. Next, the operations of these optical systems will be explained in more detail with reference to FIGS. 3A, 3B, 4A, and 4B. Note that manual control to be described below is equivalent to user's control on, e.g., driving of the photographing unit, which is performed in accordance with, e.g., the inclination angle and inclination direction of the joystick as the operational input unit 21. Also, manual photograph is equivalent to an operation of manually photographing an image of an eye to be inspected by pressing the release button of, e.g., the above-described joystick.

FIGS. 3A and 3B each show an observation image on the two-dimensional image sensor 65 of the anterior ocula segment observation optical system explained with reference to FIG. 2. An image of the anterior ocula segment of the eye E to be inspected illuminated by the anterior ocula segment observation light source 105 shown in FIG. 2 is split into upper and lower halves by the prism 63, and observed on the two-dimensional image sensor 65 as shown in FIG. 3A. As shown in FIG. 3A, portions except for the pupil look white because a large amount of the reflected light of the anterior ocula segment observation light source 105 enters. On the other hand, a pupil P looks black because no reflected light enters. Accordingly, it is possible to extract the pupil P from this contrast difference, and determine the pupil position based on the detected position of the pupil P. Referring to FIG. 3A, a pupil center PO is detected from the lower pupil P of the vertically split pupils P. The driving unit formed in the base C1 is driven so that the pupil center PO thus detected is positioned in an image center O of the two-dimensional image sensor 65 shown in FIG. 3B. This operation makes it possible to automatically perform anterior ocula segment alignment as alignment between the anterior ocula segment of the eye E to be inspected and the photographing unit C2. Note that the observation image of the image sensor 65 can also be displayed on the monitor 15 via the system controller 18.

FIGS. 4A and 4B each show an observation image on the image sensor 14 which performs both moving image observation and still photograph explained with reference to FIG. 2. Split indices 22a and 22b indicate split indices projected on the pupil of the eye E to be inspected by the focusing index projector 22 of the focusing optical system.

When the anterior ocula segment of the eye E to be inspected is automatically aligned to obtain the state shown in FIG. 3B, an image shown in FIG. 4A is observed on the image sensor 14. As described previously, the focusing index projector 22 and focusing lens 12 move in the directions of the optical axes L2 and L3 in synchronism with each other under the control of the system controller 18. Also, the image sensor 14 has an optically conjugate relationship with the focusing index projector 22. Therefore, when the focusing index projector 22 is moved in the direction of the optical axis L2, the split indices 22a and 22b move as observation images on the image sensor 14, and the focusing lens 12 synchronously moves in the direction of the optical axis L3. By controlling the split indices 22a and 22b such that the state shown in FIG. 4A changes to the (aligned) state shown in FIG. 4B on the image sensor 14, the fundus Er of the eye E to be inspected can automatically be focused. The above-described arrangement for obtaining the focused state of the photographing optical system with respect to an eye to be inspected constructs the focusing unit.

As explained above, the fundus camera of this embodiment can automatically execute both the aligning operation and focusing operation. In addition, after detecting the completion of the aligning operation and focusing operation, the system controller 18 causes the photographing optical system 4 to emit light, and executes the fundus photographing operation by the image sensor 14. That is, the operator can automatically photograph an image of the fundus by the fundus camera C by pressing the photograph start switch (not shown) of the operational input unit 21. In this case, a criterion for determining the completion of the aligning operation and focusing operation is set based on the alignment position and focusing accuracy obtained by using a plurality of eyes to be inspected as models. That is, the determination criterion is so set that the aligning operation and focusing operation are completed when an index or the like falls within a predetermined allowable range from a state in which an optimum alignment position and optimum focusing accuracy are secured.

Furthermore, the image processor 17 synthesizes the photographed fundus still image with a character image, and displays the synthetic image on the monitor 15 as an image display unit.

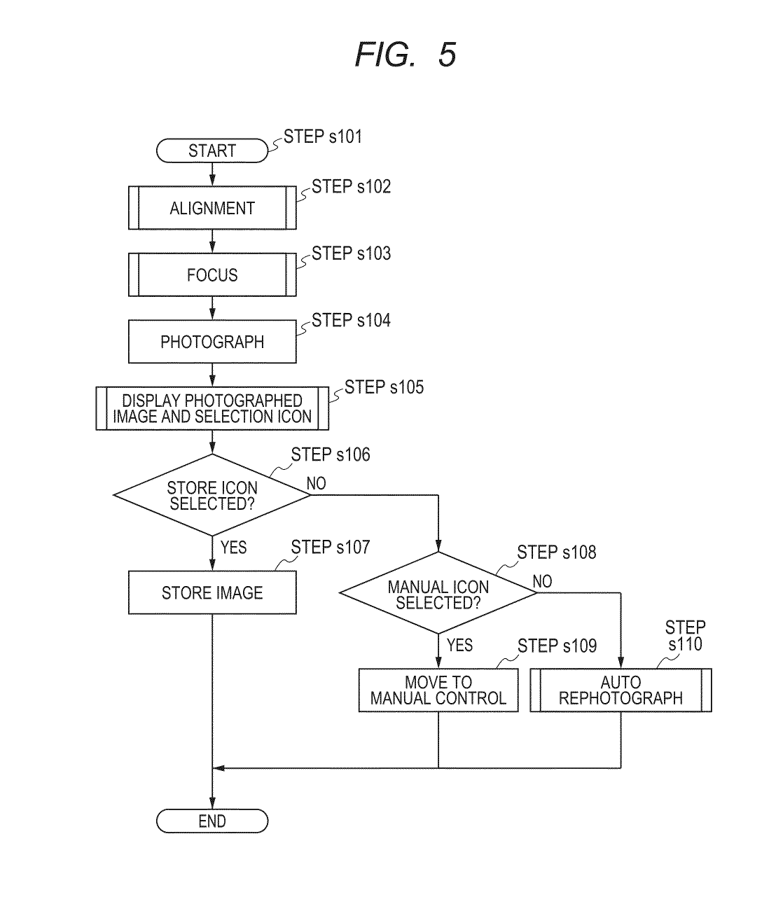

Next, a basic photographing sequence when automatically photographing an image of an eye to be inspected will be explained with reference to a flowchart shown in FIG. 5.

In step s101, an examiner performs rough alignment between a person to be inspected and the apparatus and starts a photographing operation by using various input units of the operation unit 21. In this step, the examiner causes the person to be inspected to put his or her chin on a chin receiver (not shown), and performs adjustment by using a chin receiver driving mechanism (not shown) such that the position of an eye to be inspected in the Y-axis direction becomes a predetermined height. Then, the examiner drives the photographing unit C2 by the joystick C3 to a position where the pupil of the eye E to be inspected displayed on the monitor 15 is displayed, and presses the photograph start button (not shown) after completing the driving. When the photograph start button is pressed, the system controller 18 advances the process to step s102 in order to start the operation of auto alignment.

In step s102, the anterior ocula segment and photographing unit C2 are aligned by executing the aligning operation explained with reference to FIGS. 3A and 3B. The system controller 18 determines the back-and-forth direction alignment state in the photographing unit C2 from anterior ocula segment observation images split by the prism 63. If the upper and lower halves of the observation image are shifted as shown in FIG. 3A, the system controller 18 drives a main body driving motor (not shown) in a direction in which the upper and lower halves of the observation image are not shifted as shown in FIG. 3B. Also, in alignment in the vertical and right-and-left directions, the system controller 18 detects the pupil center PO from the anterior ocula segment observation image, and drives the main body driving motor (not shown) such that the pupil center PO is positioned in the image center O of the image sensor 65. If the upper and lower halves of the anterior ocula segment observation image match and the central position and image center match, or if a shifted state within a predetermined range is obtained from the matched state, and it is determined that the alignment is complete, the process advances to step s103. Note that the image sensors 65 and 14 can perform image analysis independently of each other, so the operation of auto alignment described herein is continued even from step s103.

In step s103, focusing is performed by executing the focusing operation explained with reference to FIGS. 4A and 4B. After alignment is complete in step s102, the system controller 18 starts analyzing the output signal from the image sensor 14. Since focusing is normally not optimum in a state in which only alignment is complete, the image sensor 14 senses a fundus image in which the split indices 22a and 23b do not match as shown in FIG. 4A. Therefore, the system controller 18 controls driving of the focusing lens 12 so that the split indices 22a and 23b formed on the image sensor 14 are aligned as shown in FIG. 4B.

If it is determined that alignment and focusing are complete, the process advances to step s104. In step s104, the system controller 18 retracts the focusing index projector 22 from the optical axis L2 by a motor (not shown), and, when the retraction is complete, causes the photographing light source 4 to emit light and photographs an image of the fundus Er of the eye E to be inspected. After this photograph, the process advances to step s105, the system controller 18 causes the display monitor 15 to display the still image photographed in step s105 and an icon as a selecting unit for an examiner.

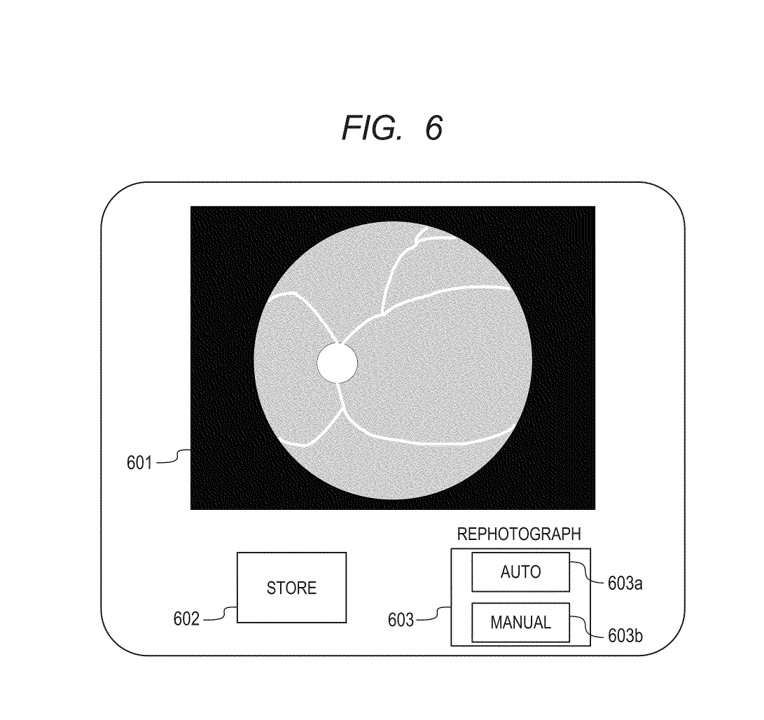

FIG. 6 shows a screen displayed on the monitor 15 as a feature of this embodiment. This screen displays a photographed still image 601, a “store” selecting icon 602 as an examiner selection input unit, and a rephotograph selecting icon 603. The rephotograph selecting icon displays an “auto” selecting icon 603a and “manual” selecting icon 603b for determining the condition of rephotograph. In step s106, the examiner is urged to determine the quality of the still image. If the examiner determines that the displayed still image 601 is worth being stored, the examiner selects the “store” icon 602. In this case, the examiner can perform this selection by pressing an icon of a touch panel sensor arranged on the screen, or by pressing a separately prepared dedicated button. If the examiner selects “store”, the process advances to step s107. In step s107, the image is stored in the storage memory 41, and the photograph is terminated. Note that the eye-to-be-inspected fundus image obtained by the photographing optical system, the above-described icons, and the like are displayed by a module which functions, in the system controller 18, as a display control unit for displaying these images on the monitor 15 as a display unit.

Also, if the examiner selects an icon in the rephotograph frame in step s106, the process advances to step s108, and the system controller 18 determines which icon in the rephotograph frame is selected in step s106. The condition of rephotograph is determined in accordance with this determination result.

If the “manual” icon is selected in step s108, the system controller 18 advances the process to step s109, changes the condition of rephotograph from “auto” to “manual”, and terminates the process. If the “auto” icon is selected in step s108, the system controller 18 performs auto rephotograph in step s110, and terminates the process after that.

Since the system controller 18 always observes the anterior ocula segment by the image sensor 14, it is possible to automatically determine whether to perform rephotograph. When the examiner selects the “auto” icon, therefore, it is also possible to automatically advance to step s104 if photograph is immediately possible, e.g., if an eye to be inspected is in a mydriatic pupil state sufficient for photograph, or if it is known that a mydriatic agent is applied to the eye to be inspected. If the eye to be inspected is in a miotic pupil state, auto photograph may be performed again after, e.g., the person to be inspected is allowed to take a rest, a mydriatic pupil state is confirmed, and the process returns to step s102.

As explained above, whether to store the fundus image displayed on the monitor 15 as a display unit is input in step s106 by the arrangement which includes the icons, buttons, or the like and functions as an input unit. Also, the system controller 18 forms an automatic driving unit which automatically controls the focusing unit and alignment unit described earlier in this embodiment. This arrangement can also be recognized as a driving unit to be automatically controlled. This driving unit drives at least one of the focusing portion in the focusing unit formed in the photographing optical system, and the photographing unit including the photographing optical system. In this case, the driving unit can also be regarded as a stage for driving the focusing portion such as a focusing lens. Furthermore, the input portions for, e.g., rephotographing exemplified by the rephotograph selecting icon 603 displayed on the monitor 15 form a selecting unit for selecting whether to execute rephotograph of an eye to be inspected by automatically controlling at least one of the focusing unit and alignment unit including, e.g., buttons by the automatic driving unit, or by manually controlling these units. If the fundus image is not to be stored, the selecting unit may also select whether to manually photograph an image of the fundus, or perform image rephotograph under the driving control conditions of the photographing optical system by which the fundus image is photographed. That is, if the displayed fundus image is not to be stored, the selecting unit may select, in accordance with a user's operation, whether to rephotograph the fundus by manually controlling the photographing optical system, or by automatically controlling the photographing optical system. This selection may also be performed based on the displayed fundus image.

In the present invention as explained above, an examiner determines the quality of a photographed image and the condition of rephotograph. This is so because as the automation of apparatuses advances, it has become difficult to automatically determine the occurrence of an imaging error by determining only the presence/absence of an abnormal light quantity of a photographed image or the occurrence of flare as in conventional systems.

The reasons of imaging errors caused by the automation of apparatuses are, e.g., defocus, flare, blink (eyelash), and an unclear fundus caused by cataract, i.e., the reasons are innumerable, and this makes automatic determination by an apparatus difficult, and may lead to a wrong diagnosis. Conversely, automatic imaging error determination is unnecessary for an examiner in many cases. Accordingly, the use of an apparatus using the flowchart of this embodiment can prevent a wrong diagnosis and raise the success probability of image rephotograph.

For example, when the reason of an imaging error is blink or eyelash, the timing of photograph is the cause of the imaging error, so a good fundus image is obtained by controlling auto alignment and auto focusing again. In a case like this, an examiner need only select “auto” photograph as the condition of repotograph. If there is defocus or flare, it is highly likely that the condition of an eye to be inspected itself does not match the functions of auto alignment and focusing of the apparatus. If photograph is performed by “auto” again, therefore, it may be possible to obtain only an identical error image. In this embodiment, however, an examiner need only select “manual” photograph. That is, when performing rephotograph, a fundus image can manually be photographed without adding any complicated automatic operation for determining an imaging error cause, e.g., stopping the automatic photographing function.

Also, when an eye to be inspected is a diseased eye such as cataract, the obtained image itself is originally unclear, so it is difficult to largely increase the clearness even by changing the photographing conditions. In this case, therefore, if an examiner determines that it is impossible to improve an image by rephotograph regardless of whether the condition is “auto” or “manual”, he or she need only select “store”. Consequently, unnecessary rephotograph can be avoided.

As described above, the present invention can provide an optimum fundus camera corresponding to an actual photographing process by presenting the condition of rephotograph to the user. As a result, unnecessary operations by an examiner can be reduced even when an imaging error has occurred.

### Second Embodiment

In the first embodiment, if flare or defocus is an imaging error cause, an examiner selects a manual operation as the condition of rephotograph. In the second embodiment, therefore, there is provided a system in which an apparatus can perform auto rephotograph by performing control parameter correction optimum for an eye to be inspected based on information obtained from a fundus image, even for flare and defocus which are most frequent causes of imaging errors.

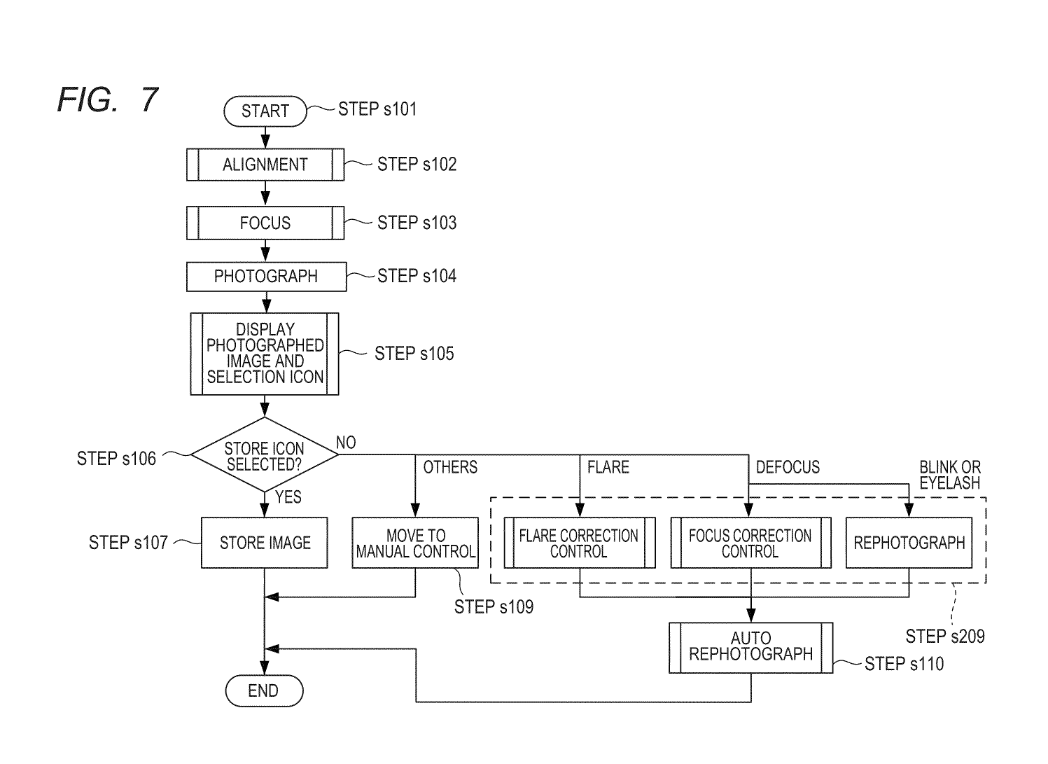

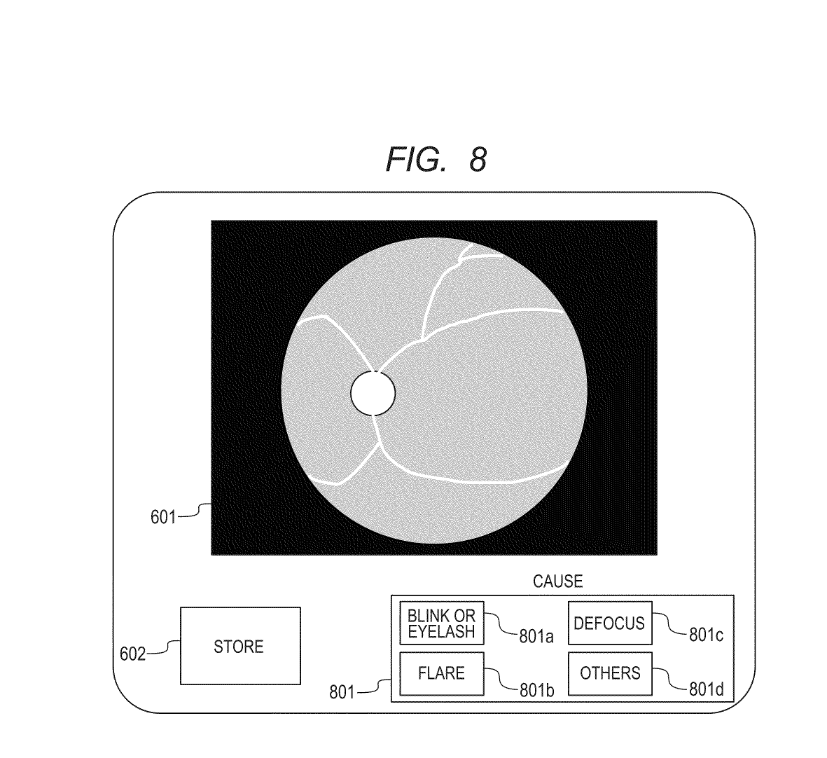

Note that the apparatus arrangement according to this embodiment is the same as that of the first embodiment, so a repetitive explanation will be omitted. An photographing sequence according to this embodiment will be explained with reference to a flowchart shown in FIG. 7 and a display screen shown in FIG. 8. Note also that in this sequence, control operations performed in steps s101 to s104 are the same as those of the first embodiment, so a repetitive explanation will be omitted.

In step s105, a system controller 18 synthesizes a photographed fundus image and selecting icons for an examiner, and displays the synthetic image on a monitor 15. FIG. 8 shows a screen displayed on the monitor 15, which is a feature of this embodiment. This screen displays a photographed still image 601, and a “store” selecting icon 602 and cause selecting icon 801 as examiner selection input units. In addition, the cause selecting icon 801 displays a list of imaging error causes as icons. In this embodiment, a “blink or eyelash” icon 801a, “flare” icon 801b, “defocus” icon 801c, and “others” icon 801d are displayed as the causes of imaging errors. Since possible causes of imaging errors are, of course, not limited to those described above, so another list of imaging error causes may also be added.

An explanation will be continued on control to be performed when the “others” icon 801d in the cause frame is selected in step s106. This control when the “others” icon in the cause list is selected is the same as that when the “manual” icon is selected in the first embodiment. If an imaging error cause does not exist in the list, the apparatus determines that it is impossible to perform auto photograph or corrected auto photograph (to be described later). In step s109, therefore, the apparatus determines that it is optimum to allow an examiner to perform operations for alignment and focusing control in rephotograph, and changes the setting of the apparatus from auto control to manual control. Next, control to be performed when an icon other than the “others” icon in the cause list is selected will be explained.

If an icon in the cause frame is selected and the selected icon is not the “others” icon 801d in step s106, the process advances to step s209. In step s209, the system controller automatically performs alignment and focusing control in rephotograph. If the “blink or eyelash” icon 801a is selected, the flow directly advances to step s110 in the same manner as when the “auto” icon is selected in the first embodiment. Then, rephotograph is performed under the same alignment condition and same focusing condition, i.e., the same driving control condition in the same manner as in the processing performed in step s110 described previously. Also, if the “flare” icon 801b or “defocus” icon 801c is selected, “flare correction” or “focus correction” (to be described later) is applied to conventional automatic control. After a correction parameter is determined in step s209, the process advances to step s110, and rephotograph is performed.

In this embodiment, if the execution of rephotograph is selected, the cause selecting icon 801 presents the causes of an imaging error as a cause list to the examiner. That is, the cause selecting icon 801 forms a presenting unit in this embodiment. At the same time, the arrangement exemplified in the cause selecting icon 801 also functions as a selecting unit for selecting an imaging error cause in order to determine a style for controlling the automatic driving unit in the above-described rephotograph. Note that the form of the selecting unit is not limited to the icon, and may also be a button form or the like as described previously. Note also that the selecting unit of this embodiment can perform selection even when rephotograph is performed under the control condition of the photographing optical system, which changes at least one of the control condition of alignment between the photographing optical system and an eye to be inspected, and the control condition of the focusing unit of the photographing optical system with respect to the fundus, in the control condition of the photographing optical system.

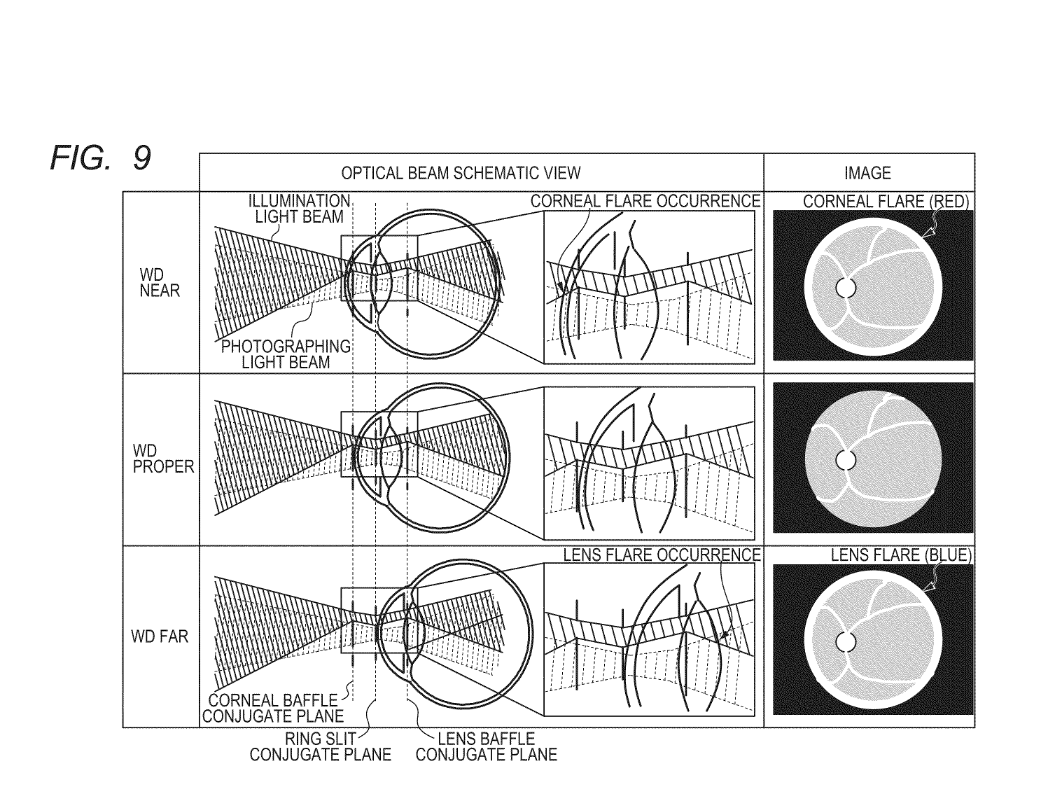

First, flare correction will be explained below. FIG. 9 is a schematic view for explaining a mechanism of causing flare as an imaging error cause. FIG. 9 shows illumination light beams (hatched portions in FIG. 9) when the working distance (to be referred to as the WD hereinafter) as a distance between a fundus camera housing corresponding to the photographing unit C2 described earlier and the eye E to be inspected changes, photographing light beams (alternate long and short dashed lines in FIG. 9), and corresponding fundus images.

In an optical beam view when the WD is an appropriate distance, images of the illumination light beams having passed through a ring slit 7, lens baffle 31, and corneal baffle 32 are formed on the conjugate planes of these members, thereby forming a light beam shown in FIG. 9. Accordingly, the illumination light beam does not overlap the photographing light beam from the cornea to the lens of the eye E to be inspected, so no flare occurs.

When the WD becomes near, however, the cornea enters a region where the illumination light beam and photographing light beam overlap. In this state, a portion of the illumination light is reflected by the cornea, and this reflected light enters the photographing light beam, so corneal flare occurs. That is, when the WD becomes near, the long-wavelength side of the illumination light beam overlaps the photographing light beam, so the color of flare becomes red.

Likewise, when the WD becomes far, the rear surface of the lens enters a region where the illumination light beam and photographing light beam overlap. Consequently, a portion of the illumination light is reflected by the lens rear surface, and this reflected light enters the photographing light beam, so lens flare occurs. That is, when the WD becomes far, the short-wavelength side of the illumination light beam overlaps the photographing light beam, so the color of flare becomes blue.

As described above, it is possible to determine the direction of misalignment from the eye to be inspected in the optical-axis direction, based on the color information of flare of the photographed fundus image. Also, the fundus camera introduced in this embodiment is designed to have a predetermined amount of margin so no flare occurs, with respect to an eye to be inspected as a design criterion. That is, letting “DC” be a margin to the cornea side with respect to an optimum WD, and “DL” be a margin to the lens side with respect to the optimum WD, the total of regions where no flare occurs can be represented by DC+DL.

An explanation will be continued on flare correction control performed in this embodiment based on the foregoing. If the “flare” icon 801b is selected as an imaging error cause in step s106, the WD may be too near or too far from the designed optimum distance. Therefore, correction of auto alignment is necessary because the same flare occurs if an image of the eye to be inspected is photographed under the same alignment condition. As described above in the explanation of FIGS. 3A and 3B, the completion of alignment between an eye to be inspected and the apparatus is determined based on whether the anterior ocula segment images vertically split by a prism 63 match. Accordingly, correction corresponding to this determination condition is performed on an eye to be inspected for which an imaging error has occurred. This makes auto alignment causing no imaging error possible.

For example, if blue flare occurs in a photographed fundus image, this means that the WD between the apparatus and an eye to be inspected is too far as described above. In rephotograph, therefore, the WD need only be made shorter than the designed optimum value. If flare is red, the WD need only be made longer than the designed optimum distance by a predetermined distance. In this embodiment, this distance to be corrected is defined as a half of a designed margin within which no flare occurs. That is, letting WD1 be an optimum alignment distance for an ideal eye to be inspected, and WD2 be an optimum alignment position after flare correction, the optimum alignment position after flare correction can be represented by:

When flare is blue: WD2=WD1−(DC+DL)/2

When flare is red: WD2=WF1+(DC+DL)/2

The prism 63 is so designed that the upper and lower images match when WD=WD1. Also, the change amount of the WD and the shift amount of the split images of the eye to be inspected can uniquely be derived by optical design. That is, the shift amount of the upper and lower images on the anterior ocula segment, which corresponds to (DC+DL)/2, can be calculated as a designed value, and the amount is a predetermined value DP. In other words, when flare correction is set in step s209, the determination condition of alignment completion in rephotograph is changed from the matching state of the upper and lower images to positions shifted to the left and right by the DP in accordance with the color of flare.

That is, when an imaging error cause selected by the selecting unit is flare, the automatic driving unit which automatically controls the alignment unit calculates the amount of misalignment between the eye to be inspected and photographing optical system from the width of flare in the periphery of a fundus image having caused an imaging error, and calculates the driving amount of the photographing unit in auto control. As for the correction amount of alignment as the distance between the eye to be inspected and photographing optical system, i.e., the driving amount, the alignment unit is automatically controlled such that the relative distance between the eye to be inspected and photographing optical system is increased when the color of flare is red, and decreased when the color is blue.

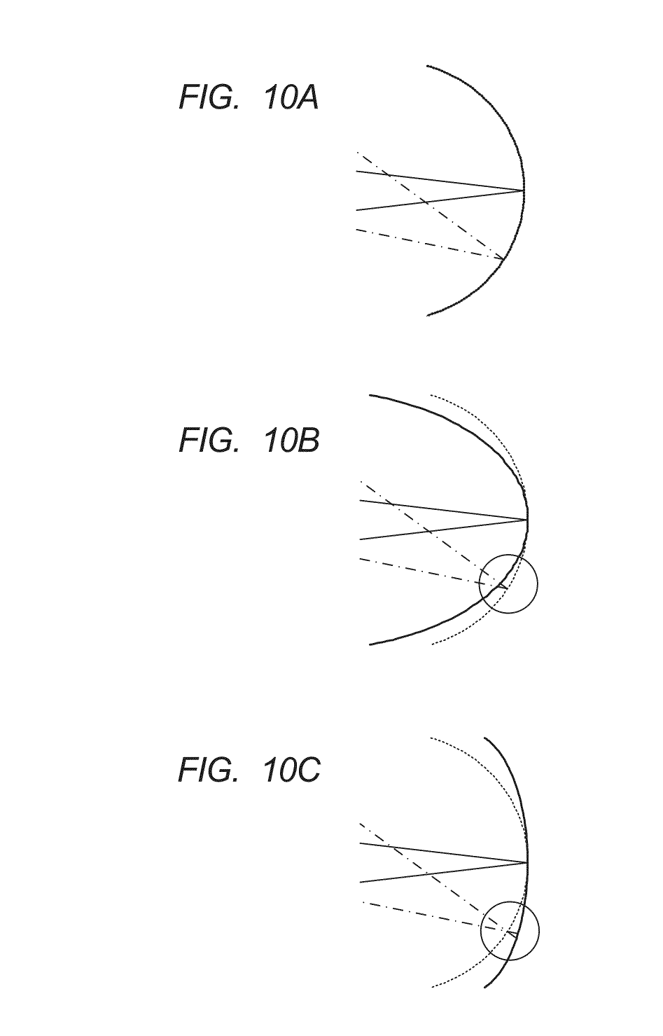

Focus correction will now be explained. FIGS. 10A to 10C are schematic views for explaining a mechanism of causing defocus as an imaging error cause. That is, FIGS. 10A, 10B, and 10C are schematic sectional views showing an ideal normal eye, an eye having a long eye axial length, and an eye having a short eye axial length, respectively. In each drawing, the thick line represents the sectional shape of the fundus, the thin line represents a light beam in the central portion of the fundus, and the alternate long and short dashed line represents a light beam in the periphery of the fundus. Also, the dotted line in each of FIGS. 10B and 10C represents the fundus sectional shape of the normal eye shown in FIG. 10A as a reference. As indicated by the thin line and alternate long and short dashed line in FIG. 10A, the optical system of the fundus camera is so designed as to optimally focus on the fundus posterior part central portion and fundus periphery. Therefore, in the extreme axial myopia shown in FIG. 10B or extreme axial hypermetropia shown in FIG. 10C, the curvature of the fundus deviates from the designed tolerance, so it is impossible to focus on the fundus posterior part center and fundus periphery.

Also, when using the focusing mechanism explained with reference to FIGS. 4A and 4B, the split indices 22a and 22b are projected onto the fundus posterior part center. Accordingly, if the focusing lens 12 is controlled such that the split indices are aligned, only the fundus posterior part is focused in the fundus image, and the fundus periphery required by the examiner is defocused (a portion enclosed with ◯ in each of FIGS. 10B and 10C is defocused).

As described in the explanation of FIGS. 4A and 4B, the split index images 22a and 22b are reflected images from the fundus. Therefore, optical design is made such that an image is formed before the fundus when the split index image 22a is positioned above the split index image 22b, formed behind the fundus when the split index image 22a is positioned below the split index image 22b, and formed on the fundus when the split indices 22 are aligned.

For the eye to be inspected having a long eye axial length as in the case shown in FIG. 10B, the split index images must be formed before the fundus posterior part in order to focus on the fundus periphery. By contrast, in the case shown in FIG. 10C, the split index images must be formed behind the fundus. However, it is difficult to determine whether the eye axial length is long or short from the photographed fundus image. In this embodiment, therefore, the eye axial length is estimated by measuring a diopter D of the eye to be inspected from the position of the focusing lens 12.

Most myopia is generally axial myopia. Therefore, if defocus occurs in an eye to be inspected having a minus diopter, the state shown in FIG. 10B has perhaps occurred. On the other hand, the state shown in FIG. 10C has probably occurred in an eye to be inspected having a plus diopter.

An explanation will be continued on focus correction control performed in this embodiment based on the foregoing. If the “defocus” icon 801c is selected as an imaging error cause in step s209, this means that the position of the focusing lens 12 is not optimum for the eye to be inspected. Accordingly, defocus similarly occurs if an image of the same eye to be inspected is photographed under the same focusing condition again. In step s110, therefore, the system controller 18 performs focus correction on the automatic focusing mechanism. This focus correction determines whether the eye to be inspected is myopia or hypermetropia from the position of the focusing lens 12 when the error image is photographed. That is, the image formation position is corrected in accordance with the value of the diopter D obtained from the position of the focusing lens 12. For example, when the diopter of the eye to be inspected is −5D, control is so performed as to form an image forward by 0.2D. When the image formation positions of the focusing indices 22 are determined, the shift amount can also be uniquely obtained as a designed value. For example, when an image must be formed forward by 0.2D as described above, correction need only be performed by ¼ the width of the focusing indices 22. That is, focus correction is set in step s110, the determination condition of focusing completion in rephotograph is changed from the state in which the focusing indices 22 are aligned to the position shifted by the correction amount.

That is, when the imaging error cause selected by the selecting unit is a defocus shift such as defocus, the automatic driving unit for automatically controlling the focusing unit acquires the diopter of the eye to be inspected from the amount of the defocus shift in rephotograph, and automatically controls the focusing unit in accordance with the diopter.

As described above, the present invention can provide an image having a little imaging error in rephotograph by presenting an imaging error cause list to the user, and causing the user to select an imaging error cause. That is, in the present invention, the examiner can freely determine the rephotograph condition after auto photograph has failed. In addition, an auto control parameter is optimally set from an imaging error cause for each eye to be inspected. Accordingly, the imaging error probability in auto rephotograph decreases, so the load to be applied on the examiner and the person to be inspected by repotograph can be reduced.

Note that in the above-described embodiment, the examiner or user selects an imaging error cause. However, an arrangement for automatically specifying an image error cause based on the feature of a fundus image having caused an imaging error described in the above explanation may also be installed in the controller 18. In this case, the controller 18 preferably includes a unit which, when the selecting unit selects an imaging error cause, changes a photographing condition when photographing a fundus image which is not stored to a photographing condition for solving the selected cause or a specified cause. The controller 18 executes rephotograph of the fundus of an eye to be inspected by using the changed photographing condition.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™, a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-208722, filed Oct. 10, 2014,which is hereby incorporated by reference wherein in its entirety.

What is claimed is:

1. An ophthalmic apparatus comprising:
a display control unit configured to cause a display unit to display a fundus image of an eye to be inspected, which is obtained by photographing an image of a fundus of the eye to be inspected by using a photographing optical system;
an input unit configured to input whether to store the displayed fundus image; and
a selecting unit configured to select, if it is input that the displayed fundus image is not to be stored, whether to execute rephotograph after automatically controlling a driving unit for driving at least one of a focusing portion formed in the photographing optical system and an photographing unit including the photographing optical system, or to execute rephotograph after manually controlling the driving unit.

2. An apparatus according to claim 1, wherein
the selecting unit includes a presenting unit configured to present an imaging error cause list to an examiner, if it is selected to execute rephotograph of the eye to be inspected by automatically controlling the driving unit for driving at least one of the focusing portion and the photographing unit in the rephotograph, and
the selecting unit executes the automatic control in accordance with selection of an imaging error cause due to which the fundus image has an imaging error, from the imaging error cause list presented by the presenting unit.

3. An apparatus according to claim 2, wherein in the automatic control, if the selected imaging error cause is flare, driving of the photographing unit by the driving unit is automatically controlled in rephotograph, and, if the selected imaging error cause is defocus shift, driving of the focusing portion by the driving unit is automatically controlled in rephotograph.

4. An apparatus according to claim 3, wherein in the automatic control, a driving amount by which the driving unit drives the photographing unit is calculated from a width of flare in a periphery of the fundus image having the imaging error, and the driving unit is automatically controlled by a driving amount in a direction which increases a relative distance between the eye to be inspected and the photographing unit when a color of the flare is red, and automatically controlled by a driving amount in a direction which decreases the relative distance when the color of the flare is blue.

5. An apparatus according to claim 4, wherein in the automatic control, if the selected imaging error cause is defocus shift, a diopter of the eye to be inspected is obtained from an amount of the defocus shift in rephotograph, and the driving unit for driving the focusing portion is automatically controlled in accordance with the diopter.

6. An ophthalmic apparatus comprising:
an input unit configured to input whether to store a fundus image of an eye to be inspected, which is obtained by photographing an image of a fundus of the eye to be inspected by using a photographing unit including a photographing optical system, and
a selecting unit configured to select, if it is input that the fundus image is not to be stored, whether to manually photograph an image of the fundus by using the photographing unit, or to rephotograph the fundus under a driving control condition of the photographing unit when the image of the fundus is photographed.

7. An apparatus according to claim 6, wherein the selecting unit can select to change, when performing rephotograph, at least one of an alignment state between the photographing unit and the eye to be inspected, and a focusing state of a focusing portion with respect to the fundus, as the driving control condition of the photographing unit.

8. An apparatus according to claim 6, wherein the driving control condition includes an alignment state between the photographing unit and the eye to be inspected, and a focusing state of a focusing portion of the photographing optical system with respect to the eye to be inspected.

9. An ophthalmic apparatus comprising:
a display control unit configured to cause a display unit to display a fundus image obtained by photographing an image of a fundus of an eye to be inspected by using a photographing unit including a photographing optical system; and
a selecting unit configured to select whether to rephotograph the fundus by manually controlling the photographing unit by a user, or to rephotograph the fundus by automatically controlling the photographing unit, in accordance with an operation performed by the user after the fundus image is displayed on the display unit.

10. An ophthalmic apparatus comprising:
a selecting unit configured to select a cause of an imaging error of a fundus image obtained by photographing an image of a fundus of an eye to be inspected by using a photographing unit including a photographing optical system; and
a control unit configured to control the photographing unit to rephotograph the fundus by using a photographing condition of solving the selected cause.

11. An apparatus according to claim 10, further comprising a display control unit configured to cause a display unit to display the fundus image,
wherein the selecting unit selects the cause in accordance with an operation performed by a user after the fundus image is displayed on the display unit.

12. An apparatus according to claim 10, wherein the selecting unit selects the cause by analyzing the fundus image.

13. An ophthalmic apparatus control method comprising steps of:
causing a display unit to display a fundus image of an eye to be inspected, which is obtained by photographing an image of a fundus of the eye to be inspected by using a photographing optical system;
inputting whether to store the displayed fundus image; and
selecting, if it is input that the displayed fundus image is not to be stored, whether to execute rephotograph after automatically controlling a driving unit for driving at least one of a focusing portion formed in the photographing optical system and a photographing unit including the photographing optical system, or to execute rephotograph after manually controlling the driving unit.

14. An ophthalmic apparatus control method comprising steps of:
inputting whether to store a fundus image of an eye to be inspected, which is obtained by rephotographing a fundus of the eye to be inspected by using a photographing unit including a photographing optical system, and
selecting, if it is input that the fundus image is not to be stored, whether to manually photograph an image of the fundus by using the photographing unit, or to rephotograph the fundus under a driving control condition of the photographing unit when the image of the fundus is photographed.

15. An ophthalmic apparatus control method comprising steps of:
causing a display unit to display a fundus image obtained by photographing an image of a fundus of an eye to be inspected by using a photographing unit including a photographing optical system; and
selecting whether to rephotograph the fundus by manually controlling the photographing unit by a user, or to rephotograph the fundus by automatically controlling the photographing unit, in accordance with an operation performed by the user after the fundus image is displayed on the display unit.

16. An ophthalmic apparatus control method comprising steps of:
selecting a cause of an imaging error of a fundus image obtained by photographing an image of a fundus of an eye to be inspected by using a photographing unit including a photographing optical system; and
controlling the photographing unit to rephotograph the fundus by using a photographing condition of solving the selected cause.
